(19) United States
(12) Patent Application Publication
(10) Pub. No.: US 2024/0324946 A1
(43) Pub. Date: Oct. 3, 2024

(54) METHOD AND DIGITAL PRODUCT SYSTEM FOR COGNITIVE IMPAIRMENT RISK PREDICTION AND PRECISE COGNITIVE TRAINING

(71) Applicant: IDEABUS TECHNOLOGY LIMITED LIABILITY COMPANY, Taichung City (TW)

(72) Inventor: Tsui-E LIN, Taichung City (TW)

(73) Assignee: IDEABUS TECHNOLOGY LIMITED LIABILITY COMPANY, Taichung City (TW)

(21) Appl. No.: 18/192,457

(22) Filed: Mar. 29, 2023

(51) Int. Cl. A61B 5/00; G16H 10/60; G16H 20/70

(52) U.S. Cl. CPC A61B 5/4088; A61B 5/6801; G16H 10/60; G16H 20/70

(57) ABSTRACT

A digital product system for predicting risk of cognitive impairment and precisely cognitive training is disclosed. The digital product system is principally composed of a hand-eye coordination training device, a brain training application program, a wearable electronic device, and a cloud computing device, so as to include multiple functions of user data collecting, cloud data analytics, risk prediction of cognitive impairment, recommendation of individual training course. Therefore, the digital product system can be adopted for conducting a cognitive function test to a subject with high testing accuracy, and providing a precise cognitive training course to the subject who has completed the cognitive function test, so as to efficiently assist the subject in enhancement of cognitive ability. In addition, the digital product system can also be utilized for improving the symptoms in a patient with Parkinson's disease, mental illness, ADHD, or ASD.

### BACKGROUND OF THE INVENTION

### 1. Field of the Invention

The present invention relates to the technology field of cognitive function improving, and more particularly to a composite electronic system embedded with AI algorithm. This composite electronic system includes multiple functions of user data collecting, cloud data analytics, risk prediction of cognitive impairment, recommendation of individual training course, such that the composite electronic system can be utilized for conducting a cognitive function test to a subject with high testing accuracy, and providing a precise cognitive training course to the subject who has completed the cognitive function test, so as to efficiently assist the subject in enhancement of cognitive ability.

### 2. Description of the Prior Art

Population ageing refers to the rise in life expectancy and the fall in birth rates, which together cause the age group of people over 60 to grow as a proportion of the entire population. Therefore, the age structure of the elderly population in Taiwan is also growing rapidly, and the incidence of dementia is gradually increasing. Therefore, how to easily and objectively evaluate the cognitive function of the elderly has become an urgent need. It is known that, mini-mental state examination (MMSE) or Folstein test is a 30-point questionnaire that is used extensively in clinical and research settings to measure cognitive impairment. In general, in case of the fact that a subject gets a MMSE score less than 16 points after completing the Folstein test, the subject is diagnosed with cognitive impairment.

Dementia is typically diagnosed when acquired cognitive impairment has become severe enough to compromise daily living skills, social ability and/or occupational functioning. Dementia is a general term for loss of memory, language, problem-solving and other thinking abilities that are severe enough to interfere with daily life. Therefore, clinic experiences indicates that, designing the MMSE test to become an interactive game is helpful in stimulating and activating the subject who is receiving the MMSE test by playing the interactive game.

Cognitive training appeared to optimize cognitive functions in patient elderly diagnosed with MCI, such that it may have the potential to slow the rate of cognitive and functional decline. However, if related reports of medical laboratory have determined a treatable medical condition as the cause of the mild cognitive impairment, the patient should be treated for those conditions. Also, medications may be prescribed if behavioral or psychiatric symptoms (for example, agitation, anger, anxiety, sleep problems, depression, delirium) are present and interfering with the patient's quality of life.

The real condition is that, after being diagnosed with cognitive impairment, what the patient can do is to get drugs for correspondingly curing behavioral disturbances and/or psychotic symptoms according to prescriptions wrote by his doctor, or is to do rehabilitation exercises including hand-eye coordination training and brain activation training according to doctor's suggestions. Apparently, regularly going to hospital based on follow-up appointment is inconvenient for the physically challenged patient and the patient living in health workforce shortage areas. It is imaginable that, state of the cognitive impairment certainly gets worse if the patient fails to regularly go to hospital based on follow-up appointment or seek a proper medical treatment in time.

According to above descriptions, it is understood that there are still rooms for improvement in the conventional cognitive function testing and cognitive impairment treating. In view of this fact, inventors of the present application have made great efforts to make inventive research and eventually provided a method and digital product system for cognitive impairment risk rediction and precise cognitive training.

### SUMMARY OF THE INVENTION

The primary objective of the present invention is to disclose a method and digital product system for cognitive impairment risk rediction and precise cognitive training. According to the present invention, the digital product system is a composite electronic system embedded with AI algorithms, and is composed of a hand-eye coordination training device, a brain training application program, a wearable electronic device, and a cloud computing device, such that the digital product system includes multiple functions of user data collecting, cloud data analytics, risk prediction of cognitive impairment, recommendation of individual training course. Therefore, the digital product system can be adopted for conducting a cognitive function test to a subject with high testing accuracy, and providing a precise cognitive training course to the subject who has completed the cognitive function test, so as to efficiently assist the subject in enhancement of cognitive ability. In addition, the digital product system can also be utilized for improving the symptoms in a patient with Parkinson's disease, mental illness, attention deficit hyperactivity disorder (ADHD), or autism spectrum disorder (ASD).

Moreover, by operating the hand-eye coordination training device, the brain training application program, and the wearable electronic device, the patient is able to attend an on-line cognitive function test, at least one precise cognitive training course, an evaluation of the risk of cognitive impairment, and an analysis of the training effect, thereby improving the symptoms thereof. Particularly, during conducting the precise cognitive training course, the composite electronic system (i.e., digital product system) improve the patient's body function and brain activity by enhancing the sense of hand-eye coordination, flexibility and/or balance. Therefore, after using this digital product system for a period of time, the patient's daily living skills, social ability and/or occupational functioning would be significantly recovered because having enforced reaction, memory, attention, logic, and physical coordination. As a result, the patient's quality of life is elevated.

As described in more detail below, the main framework for predicting risk of cognitive impairment and precisely cognitive training consists of IoT device, application program, cloud data analyzing device, AI algorithm for predicting risk of cognitive impairment, and cognitive training course with personalization. In which, the IoT device and the application program are adopted for collecting the data of the cognitive function test and the physical data monitored during the patient receiving the cognitive training. Therefore, based on the collected test data and physical data, the AI algorithm can succeed in predicting risk of cognitive impairment of the patient, and then re-designs or improves the content of at least one cognitive training course. As a result, since being designed according to each individual, the cognitive training course with personalization is significantly helpful in enforcing reaction, memory, attention, logic and physical coordination of the patient.

The digital product system is a composite electronic system embedded with AI algorithm, and is composed of a tablet computer (or a personal computer) and multiple smart wearable electronic devices having sensors and controller, so as to be used for generating cognitive training course that is helpful in significantly enhancing the patient's cognitive and physical ability. During the cognitive training, the patient is guided to conduct a series of exercises related to cognitive and physical training. Moreover, the composite electronic system is installed with an AI program therein, such that that the composite electronic system is only able to predict risk of cognitive impairment of the patient, but also can re-design or improve the content of at least one cognitive training course according to the patient's clinic data.

More specifically, the hand-eye coordination training device is controlled to generates an interactive game in a cognitive function test. After playing the interactive game so as to complete the cognitive function test, the cloud computing device immediately calculates a MMSE score for the subject. Subsequently, the cloud computing device intelligently recommends a suitable cognitive training course to the subject, such that the subject is able to conduct the cognitive training course in case of wearing the wearable electronic device. Moreover, during the subject conducting the cognitive training course, the wearable electronic device monitors the subject's training data by the sensors thereof, and then uploads the training data to the cloud computing device, such that the cloud computing device succeeds in evaluation of the subject's action accuracy, physical expansion, physical balance, and agility. As such, the cloud computing device is able to select a specific training course consisting of at least one training exercise from a database according to the MMSE score, the test data and the training data, thereby recommending the training course to the subject. Consequently, conducting the at least one training exercise is helpful in improving the subject's cognitive abilities.

In addition, the cloud computing device is further provided with a machine learning model constituted by artificial neural networks (ANN), and the machine learning model can be trained so as to become a training course recommending model. As such, after calculating a MMSE score for the subject, the cloud computing device selects a training course from a database by executing the training course recommending model, thereby recommending the training course to the subject.

In one aspect of the invention, a digital product system for predicting risk of cognitive impairment and precisely cognitive training is disclosed. The digital product system comprises: a subject-end tablet computer, an interactive cognitive training apparatus (Me-SODA), a wearable electronic device, and a cloud computing device. After a test game application program being launched, the tablet computer controls the interactive cognitive training apparatus to generate an interactive game in a cognitive function test. After playing the interactive game so as to complete the cognitive function test, the cloud computing device immediately calculates a MMSE score for the subject. Subsequently, the cloud computing device intelligently recommends a suitable cognitive training course to the subject, such that the subject is able to conduct the cognitive training course in case of wearing the wearable electronic device. Moreover, during the subject conducting the cognitive training course, the wearable electronic device monitors the subject's training data by the sensors thereof, and then uploads the training data to the cloud computing device, such that the cloud computing device is able to select a specific training course consisting of at least one training exercise from a database according to the MMSE score, the test data and the training data, thereby recommending the training course to the subject. Consequently, conducting the at least one training exercise is helpful in improving the subject's cognitive abilities. Therefore, after using this digital product system for a period of time, the patient's daily living skills, social ability and/or occupational functioning would be significantly recovered because having enforced reaction, memory, attention, logic, and physical coordination. As a result, the patient's quality of life is elevated.

In addition, the digital product system of the present invention further comprises a therapist-end tablet computer. More specifically, a therapist can be chosen as a counselor for a subject, and is allowed to, by operating the tablet computer, obtain the test data and the training data of the subject from the cloud computing device. As such, the therapist is able to evaluate the cognitive ability and the training effect of the subject, so as to decide to re-design or improve the content of at least one cognitive training course, thereby uploading the redesigned cognitive training course and/or the improved cognitive training course to a training course database of the cloud computing device.

In brief, the digital product system of the present invention is not only able to conduct a cognitive function test to a subject so as to predict the risk of cognitive impairment, and is also capable of intelligently recommending a suitable cognitive training course to the subject. Therefore, after conducting the cognitive training course for a period of time, the subject is not only well trained in cognitive abilities of reaction, memory, attention, logic, and physical coordination, and is also well trained in physical abilities of sensitivity, muscular endurance and agility. As a result, the patient's daily living skills, social ability and/or occupational functioning would be significantly recovered, so as to make the patient's quality of life be elevated.

For achieving the primary objective mentioned above, the present invention provides an embodiment of the digital product system, which is adopted for conducting a cognitive function test to a subject, and is also used for providing at least one cognitive training course to the subject. The digital product system comprises:
a cloud computing device;
a first electronic device in communication with the cloud computing device, being provided to the subject to operate;
a second electronic device in communication with the first electronic device; and
a wearable electronic device in communication with the first electronic device;
wherein the first electronic device is configured for controlling the second electronic device to conduct a cognitive function test to the subject, so as to collect a test data in the cognitive function test, thereby transmitting the test data to the cloud computing device;
wherein the cloud computing device is configured to select at least one cognitive training course from a database according to the test data and a user parameter of the subject, and then recommends the cognitive training course to the subject through the first electronic device, such that the subject is able to conduct the cognitive training course by operating the first electronic device and/or the wearable electronic device.

In one embodiment, the first electronic device is selected from a group consisting of tablet computer, smart phone, smart television, laptop computer, desktop computer, and all-in-one computer.

In one embodiment, the user parameter comprises at least one selected from a group consisting of age, BMI, gender, education level, and MMSE score.

In one embodiment, the second electronic device is selected from a group consisting of gaming device, tablet computer, virtual reality helmet, and mixed reality helmet.

In one embodiment, the first electronic device comprises a first processor and a first memory storing a plurality of application programs, in which the plurality of application programs comprise:
a first application program, including instructions for configuring the first processor to control the second electronic device to conduct a cognitive function test to the subject, so as to collect the test data in the cognitive function test;
a second application program, including instructions for configuring the first processor to control the wearable electronic device to collect a training data in case of the subject wearing the wearable electronic device to conduct the cognitive training course; and
a third application program, including instructions for configuring the first processor to control the first electronic device and/or the second electronic device to generate a specific game helpful in stimulating and activating a brain of the subject.

In a practicable embodiment, the digital product system further comprises:
a third electronic device in communication with the cloud computing device, being provided to a professional personnel to operate;
wherein the third electronic device is configured for receiving the test data and the training data from the cloud computing device;
wherein by operating the third electronic device, a content of one cognitive training course is able to be redesigned or improved; and
wherein by operating the third electronic device, the redesigned cognitive training course and/or the improved cognitive training course are transmitted to the cloud computing device, so as to be store in the database.

In one embodiment, the third electronic device is selected from a group consisting of tablet computer, smart phone, smart television, laptop computer, desktop computer, and all-in-one computer.

In one embodiment, the cloud computing device comprises a processor and a memory, and a pre-trained machine learning model being stored in the memory, such that the processor is able to access the memory so as to execute the machine learning model, and then selecting at least one cognitive training course from the database according to the test data and the user parameter of the subject, thereby recommending the cognitive training course to the subject through the first electronic device.

In a practicable embodiment, the digital product system further comprises a monitoring device in communication with the first electronic device, wherein the monitoring device is configured for monitoring the subject in case of the subject is conducting the cognitive training course, so as to collect the training data for further uploading to the cloud computing device.

In one embodiment, the monitoring device comprises at least one selected from a group consisting of camera and motion capture system.

In another one practicable embodiment, the wearable electron device includes a plurality of sensors, and the wearable electron device is configured for monitoring the subject in case of the subject is conducting the cognitive training course, so as to collect the training data for further uploading to the cloud computing device.

In one embodiment, the memory of the cloud computing device further stores an application program including instructions, and the processor is configured to execute the instructions to:
input a first sample including at least one test data, a second sample including at least one training data, a third sample including at least one user parameter, and a plurality of cognitive training course samples to a machine learning model, so as to lead the machine learning model to output a predicted outcome in relation with a categorical cognitive training course; wherein each said cognitive training course sample is associated with a categorical label;
compare the predicted outcome with one corresponding categorical label, and then generate a comparison data, thereby adaptively modulating at least one model parameter of the machine learning model according to the comparison data;
input the first sample, the second sample, the third sample, and the cognitive training course samples to the post-modulation machine learning model, and then calculate an accuracy of training course recommending;
re-conduct the foregoing all steps in case of the accuracy being below a pre-determined value iteratively; and
take the post-modulation machine learning model as said pre-trained machine learning model so as to be stored in the memory after the accuracy is equal to or greater than the pre-determined value.

### BRIEF DESCRIPTION OF THE DRAWINGS

The invention as well as a preferred mode of use and advantages thereof will be best understood by referring to the following detailed description of an illustrative embodiment in conjunction with the accompanying drawings, wherein:

FIG. 1 shows a schematic stereo diagram of a digital product system for predicting risk of cognitive impairment and precisely cognitive training according to the present invention;

FIG. 2 shows a block diagram of the digital product system according to the present invention;

FIG. 3 shows a block diagram of a first electronic device that is depicted in FIG. 1; and

FIG. 4 shows a block diagram of a cloud computing device that is depicted in FIG. 1.

### DETAILED DESCRIPTION OF THE PREFERRED EMBODIMENTS

To more clearly describe a method and digital product system for cognitive impairment risk rediction and precise cognitive training according to the present invention, embodiments of the present invention will be described in detail with reference to the attached drawings hereinafter.

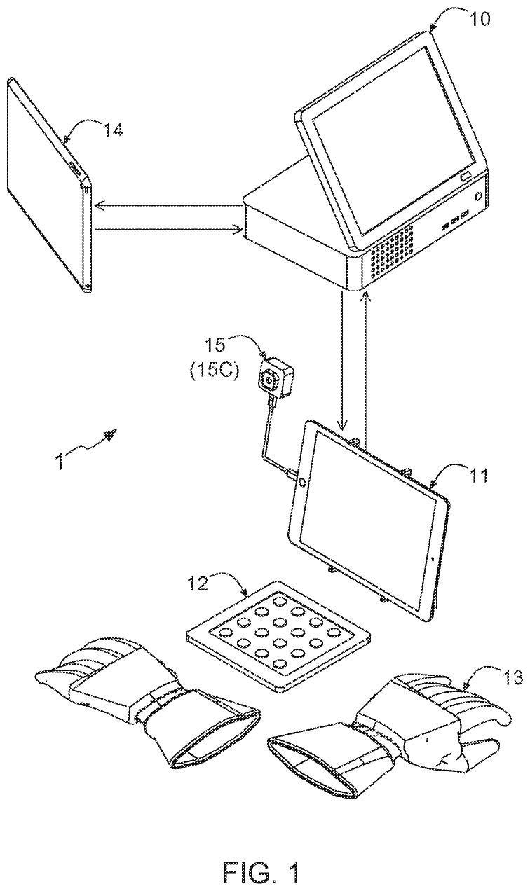

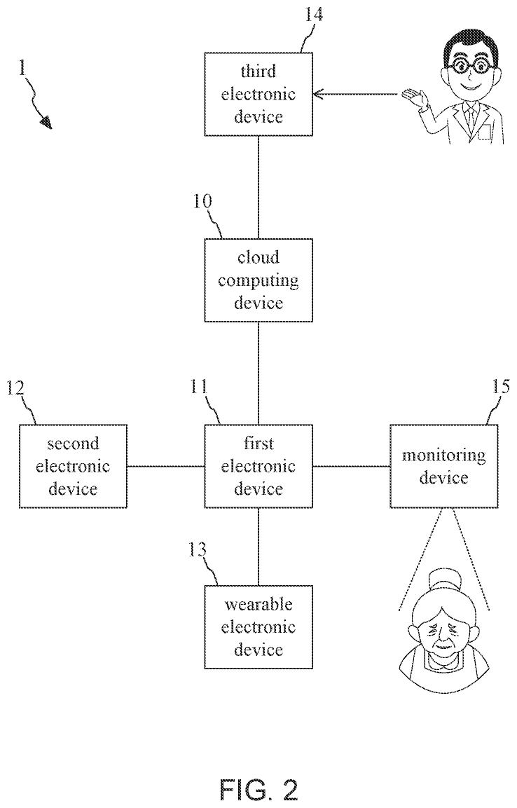

With reference to FIG. 1, there is shown a digital product system for predicting risk of cognitive impairment and precisely cognitive training according to the present invention. Moreover, FIG. 2 illustrates a block diagram of the digital product system. As FIG. 1 and FIG. 2 show, the digital product system 1 comprises: a cloud computing device 10, a first electronic device 11 (e.g., subject-end tablet computer), a second electronic device 12, a wearable electronic device 13, and a third electronic device 14 (e.g., therapist-end tablet computer). In practical application, the second electronic device 12 can be, but is not limited to, a gaming device particularly designed for hand-eye coordination training, a tablet computer, a virtual reality helmet, or a mixed reality helmet. On the other hand, the first electronic device 11 can be, but is not limited to, a tablet computer, a smart phone, a smart television, a laptop computer, a desktop computer, or an all-in-one computer (e.g., iMAC).

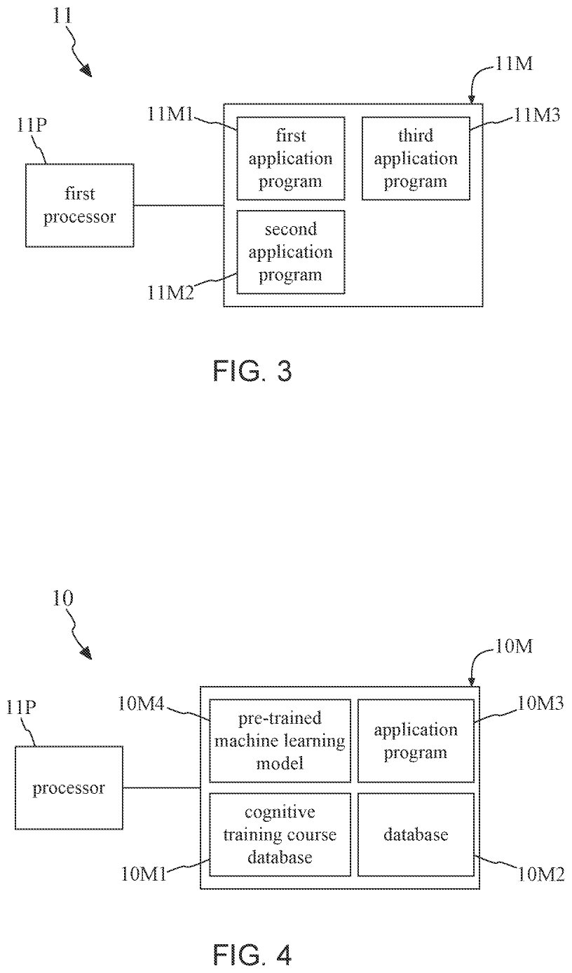

FIG. 3 shows a block diagram of a first electronic device 11 that is depicted in FIG. 1. As FIG. 1, FIG. 2 and FIG. 3 show, the first electronic device 11 is in communication with the cloud computing device 10 and the second electronic device 12, and comprises a first processor 11P and a first memory 11M, in which the first memory stores a plurality of application programs. In one embodiment, the plurality of application programs comprises a first application program 11M1, a second application program 11M2 and a third application program 11M3. According to the particularly design of the present invention, the subject is allowed to operate his tablet computer (i.e., the first electronic device 11) to launch the first application program 11M1. After the first processor 11P is commanded to access the first memory 11M so as to execute the first application program 11M1, the first processor 11P is configured to control the second electronic device 12. Therefore, in case of being controlled by the first electronic device 11, the second electronic device 12 (i.e., gaming device particularly designed for hand-eye coordination training) conducts a cognitive function test to the subject. In one embodiment, the cognitive function test is an interactive game including a plurality of game chapters, and the subject needs to play all of the game chapters for completing the cognitive function test. Particularly, it is necessary for subject to play each game chapter by using his hands and eyes coordinately, such that the brain of the subject is well stimulated so as to be activated after the subject completes the interactive game. In fact, technique details about designing the cognitive function test to an interactive game have been disclosed in the specification of Taiwan Patent No. 1791323.

The first electronic device 11 collects a test data of the subject during the cognitive function test, and then transmits the test data to the cloud computing device 10 after the cognitive function test is ended. In one embodiment, the plurality of game chapters comprises: a first game chapter related to a reaction testing, a second game chapter related to a memory testing, a third game chapter related to an attention testing, a fourth game chapter related to a physical coordination ability testing, and a fifth game chapter related to a logic testing. Therefore, the test data comprises: MMSE (Mini-mental state examination) score, count of correct answers the subject submits in the first game chapter, amount of time the subject spends in completing the first game chapter, count of correct answers the subject submits in the second game chapter, amount of time the subject spends in completing the second game chapter, count of correct answers the subject submits in the third game chapter, amount of time the subject spends in completing the third game chapter, count of correct answers the subject submits in the fourth game chapter, amount of time the subject spends in completing the fourth game chapter, count of correct answers the subject submits in the fifth game chapter, amount of time the subject spends in completing the fifth game chapter, and correct rate. Furthermore, FIG. 4 shows a block diagram of a cloud computing device that is depicted in FIG. 1. According to the present invention, the cloud computing device 10 is configured to, based on the test data and a user parameter of the subject, select at least one cognitive training course from a cognitive training course database 10M1 thereof, thereby recommending the cognitive training course to the subject through the first electronic device 11. As a result, the subject is able to conduct the cognitive training course by operating the first electronic device 11 and/or the wearable electronic device 13. In one embodiment, the user parameter comprises at least one of age, BMI, gender, education level, and MMSE score.

Subsequently, the subject is able to wear the wearable electronic device 13, and then operates the tablet computer (i.e., first electronic device 11) to play video that containing the related content of the cognitive training course. Moreover, according to the present invention, a therapist (i.e., professional personnel) can be chosen as a counselor for a subject, and is allowed to, by operating his tablet computer (i.e., third electronic device 14), obtain the test data and a training data of the subject from the cloud computing device 10. As such, the therapist is able to evaluate the cognitive ability and the training effect of the subject, so as to decide to re-design or improve the content of at least one cognitive training course that includes a first chapter related to a reaction training, a second chapter related to a memory training, a third chapter related to an attention training, a fourth chapter related to a physical coordination ability training, and a fifth chapter related to a logic training. After that, the therapist is able to thereby uploading the redesigned cognitive training course and/or the improved cognitive training course to the cognitive training course database 10M1 of the cloud computing device 10. Therefore, after conducting the cognitive training course for a period of time, the subject is not only well trained in cognitive abilities of reaction, memory, attention, logic, and physical coordination, and is also well trained in physical abilities of sensitivity, muscular endurance and agility. As a result, the subject's daily living skills, social ability and/or occupational functioning would be significantly recovered, so as to make the subject's quality of life be elevated.

In addition, the first memory further stores a second application program 11M2. As such, during the subject conducting the cognitive training course, the first processor 11P is configured to, in case of the subject wearing the wearable electronic device 13 to conduct the cognitive training course, control the wearable electronic device 13 to collect a training data by its multiple sensors. In other words, the wearable electronic device 13 is also functioned as a monitoring device in the digital product system 1 of the present invention. On the other hand, the first memory further stores a third application program 11M3. As such, the subject is allowed to launch the third application program 11M3 by operating the first electronic device 11. Therefore, the first processor 11P is configured to control the first electronic device 11 and/or the second electronic device 12 (i.e., gaming device particularly designed for hand-eye coordination training) to generate a specific game helpful in stimulating and activating a brain of the subject.

In practical application, the digital product system 1 can be designed to further include a monitoring device 15 in communication with the first electronic device 11. The monitoring device 15 can comprises at least one camera 15C and/or a motion capture system, and is configured for monitoring the subject in case of the subject is conducting the cognitive training course, so as to collect the training data for further uploading to the cloud computing device 10.

Furthermore, as FIG. 1, FIG. 2, FIG. 3, and FIG. 4 show, by operating the third electronic device 14 (i.e., therapist-end tablet computer) to be in communication with the cloud computing device 10, the therapist is able to obtain the test data and the training data of the subject from the cloud computing device 10. As such, the therapist can to evaluate the cognitive ability and the training effect of the subject, so as to decide to re-design or improve the content of at least one cognitive training course, thereby uploading the redesigned cognitive training course and/or the improved cognitive training course to the cognitive training course database 10M1 of the cloud computing device 10. In practical application, the third electronic device 14 can be, but is not limited to, a tablet computer, a smart phone, a smart television, a laptop computer, a desktop computer, or an all-in-one computer (e.g., iMAC).

On the other hand, the cloud computing device 10 comprises a processor 10P and a memory 10M, in which the memory 10M includes said Cognitive training course database 10M1 and a databased 10M2 for storing personality data including user parameters. As explained in more detail below, the therapist commonly designs at least one cognitive training course with personalization based on a specific subject's test data, training data, and user parameter comprising at least one of age, BMI, gender, education level, and MMSE score. In other words, personalized cognitive training course is associated with a subject data including the test data, the training data, and the user parameter, such that the subject data can be made to be a categorical label for remarking at least one cognitive training course.

Therefore, it is possible to design an application program 10M3 to be stored in the memory 10M of the clouding computing device 10. Then, it is allowed to launch the application program 10M3 for generated a pre-trained machine learning model 10M4. Computer science (CS) engineers skilled in design of AI program certainly know that, machine learning means computers learning from data using algorithms to perform a task without being explicitly programmed, and deep learning algorithm is a subset of machine learning technology. For example, supervised learning algorithm is developed based on deep learning, which works depends upon artificial neural networks (ANN) that consists of an input layer, at least one hidden layer and an output layer. Feed forward neural networks are artificial neural networks in which nodes do not form loops. This type of neural network is also known as a multi-layer neural network as all information is only passed forward. During data flow, input nodes receive data, which travel through hidden layers, and exit output nodes. No links exist in the network that could get used to by sending information back from the output node.

Therefore, it is possible to design a machine learning model including artificial neural networks, so as to utilize a training set consisting of a plurality of samples to train the machine learning model to form said pre-trained machine learning model 10M4 stored in the memory of the cloud computing device 10. According to the present invention, the particularly-designed application program 10M3 including instructions, and the processor 10P is configured to execute the instructions to:
input a first sample including at least one test data, a second sample including at least one training data, a third sample including at least one user parameter, and a plurality of cognitive training course samples to the foregoing machine learning model including artificial neural networks, so as to lead the machine learning model to output a predicted outcome in relation with a categorical cognitive training course; wherein each said cognitive training course sample is associated with a categorical label;
compare the predicted outcome with one corresponding categorical label, and then generate a comparison data, thereby adaptively modulating at least one model parameter of the machine learning model according to the comparison data;
input the first sample, the second sample, the third sample, and the cognitive training course samples to the post-modulation machine learning model, and then calculate an accuracy of training course recommending;
re-conduct the foregoing all steps in case of the accuracy being below a pre-determined value iteratively; and
take the post-modulation machine learning model as said pre-trained machine learning model so as to be stored in the memory after the accuracy is equal to or greater than the pre-determined value.

After launching the application program 10M3 to train the machine learning model including artificial neural networks to form a cognitive training course recommending model (i.e., pre-trained machine learning model 10M4), the cognitive training course recommending model is further stored in the memory 10M of the cloud computing device 10, such that the processor 10P is able to access the memory 10M so as to execute the cognitive training course recommending model, thereby selecting at least one cognitive training course from the cognitive training course database 10M1 according to the test data, the training data and the user parameter of the subject. Consequently, the cloud computing device 10 recommends the cognitive training course to the subject through the first electronic device 11.

Therefore, through above descriptions, all embodiments and their constituting elements of the digital product system for predicting risk of cognitive impairment and precisely cognitive training disclosed by the present invention have been introduced completely and clearly; in summary, the present invention includes the advantages of:

(1) the present invention discloses a method and digital product system for cognitive impairment risk rediction and precise cognitive training. According to the present invention, the digital product system comprises a subject-end tablet computer, an interactive cognitive training apparatus (Me-SODA), a wearable electronic device, and a cloud computing device. After a test game application program being launched, the tablet computer controls the interactive cognitive training apparatus to generate an interactive game in a cognitive function test. After playing the interactive game so as to complete the cognitive function test, the cloud computing device immediately calculates a MMSE score for the subject. Subsequently, the cloud computing device intelligently recommends a suitable cognitive training course to the subject, such that the subject is able to conduct the cognitive training course in case of wearing the wearable electronic device.

(2) Moreover, during the subject conducting the cognitive training course, the wearable electronic device monitors the subject's training data by the sensors thereof, and then uploads the training data to the cloud computing device, such that the cloud computing device is able to select a specific training course consisting of at least one training exercise from a database according to the MMSE score, the test data and the training data, thereby recommending the training course to the subject. Consequently, conducting the at least one training exercise is helpful in improving the subject's cognitive abilities. Therefore, after using this digital product system for a period of time, the subject's daily living skills, social ability and/or occupational functioning would be significantly recovered because having enforced reaction, memory, attention, logic, and physical coordination. As a result, the subject's quality of life is elevated.

(3) In addition, the digital product system of the present invention further comprises a therapist-end tablet computer. More specifically, a therapist can be chosen as a counselor for a subject, and is allowed to, by operating the tablet computer, obtain the test data and the training data of the subject from the cloud computing device. As such, the therapist is able to evaluate the cognitive ability and the training effect of the subject, so as to decide to re-design or improve the content of at least one cognitive training course, thereby uploading the redesigned cognitive training course and/or the improved cognitive training course to a training course database of the cloud computing device.

The above description is made on embodiments of the present invention. However, the embodiments are not intended to limit the scope of the present invention, and all equivalent implementations or alterations within the spirit of the present invention still fall within the scope of the present invention.

What is claimed is:

1. A digital product system, being adopted for conducting a cognitive function test to a subject, and being used for providing at least one cognitive training course to the subject; the digital product system comprising:
a cloud computing device;
a first electronic device in communication with the cloud computing device, being provided to the subject to operate;
a second electronic device in communication with the first electronic device; and
a wearable electronic device in communication with the first electronic device;
wherein the first electronic device is configured for controlling the second electronic device to conduct a cognitive function test to the subject, so as to collect a test data in the cognitive function test, thereby transmitting the test data to the cloud computing device;
wherein the cloud computing device is configured to select at least one cognitive training course from a database according to the test data and a user parameter of the subject, and then recommends the cognitive training course to the subject through the first electronic device, such that the subject is able to conduct the cognitive training course by operating the first electronic device and/or the wearable electronic device.

2. The digital product system of claim 1, wherein the first electronic device is selected from a group consisting of tablet computer, smart phone, smart television, laptop computer, desktop computer, and all-in-one computer.

3. The digital product system of claim 1, wherein the user parameter comprises at least one selected from a group consisting of age, BMI, gender, education level, and MMSE score.

4. The digital product system of claim 1, wherein the second electronic device is selected from a group consisting of gaming device, tablet computer, virtual reality helmet, and mixed reality helmet.

5. The digital product system of claim 2, wherein the first electronic device comprises a first processor and a first memory storing a plurality of application programs.

6. The digital product system of claim 5, wherein the plurality of application programs comprise:
a first application program, including instructions for configuring the first processor to control the second electronic device to conduct a cognitive function test to the subject, so as to collect the test data in the cognitive function test;
a second application program, including instructions for configuring the first processor to control the wearable electronic device to collect a training data in case of the subject wearing the wearable electronic device to conduct the cognitive training course; and
a third application program, including instructions for configuring the first processor to control the first electronic device and/or the second electronic device to generate a specific game helpful in stimulating and activating a brain of the subject.

7. The digital product system of claim 6, further comprising:
a third electronic device in communication with the cloud computing device, being provided to a professional personnel to operate;
wherein the third electronic device is configured for receiving the test data and the training data from the cloud computing device;
wherein by operating the third electronic device, a content of one cognitive training course is able to be redesigned or improved; and
wherein by operating the third electronic device, the redesigned cognitive training course and/or the improved cognitive training course are transmitted to the cloud computing device, so as to be store in the database.

8. The digital product system of claim 7, wherein the third electronic device is selected from a group consisting of tablet computer, smart phone, smart television, laptop computer, desktop computer, and all-in-one computer.

9. The digital product system of claim 7, wherein the cloud computing device comprises a processor and a memory, and a pre-trained machine learning model being stored in the memory, such that the processor is able to access the memory so as to execute the machine learning model, and then selecting at least one cognitive training course from the database according to the test data and the user parameter of the subject, thereby recommending the cognitive training course to the subject through the first electronic device.

10. The digital product system of claim 7, further comprising a monitoring device in communication with the first electronic device, wherein the monitoring device is configured for monitoring the subject in case of the subject is conducting the cognitive training course, so as to collect the training data for further uploading to the cloud computing device.

11. The digital product system of claim 10, wherein the monitoring device comprises at least one selected from a group consisting of camera and motion capture system.

12. The digital product system of claim 10, wherein the wearable electron device includes a plurality of sensors, and the wearable electron device is configured for monitoring the subject in case of the subject is conducting the cognitive training course, so as to collect the training data for further uploading to the cloud computing device.

13. The digital product system of claim 11, wherein the memory of the cloud computing device further stores an application program including instructions, and the processor is configured to execute the instructions to:
input a first sample including at least one test data, a second sample including at least one training data, a third sample including at least one user parameter, and a plurality of cognitive training course samples to a machine learning model, so as to lead the machine learning model to output a predicted outcome in relation with a categorical cognitive training course; wherein each said cognitive training course sample is associated with a categorical label;
compare the predicted outcome with one corresponding categorical label, and then generate a comparison data, thereby adaptively modulating at least one model parameter of the machine learning model according to the comparison data;
input the first sample, the second sample, the third sample, and the cognitive training course samples to the post-modulation machine learning model, and then calculate an accuracy of training course recommending;
re-conduct the foregoing all steps in case of the accuracy being below a pre-determined value iteratively; and
take the post-modulation machine learning model as said pre-trained machine learning model so as to be stored in the memory after the accuracy is equal to or greater than the pre-determined value.
